(19) United States
(12) Patent Application Publication
(10) Pub. No.: US 2023/0056088 A1
(43) Pub. Date: Feb. 23, 2023

(54) DEOXYHEMOGLOBIN IN MAGNETIC RESONANCE IMAGING

(71) Applicants: Adrian P. CRAWLEY, Toronto (CA); Rohan DHARMAKUMAR, Moorpark, CA (US); James DUFFIN, Toronto (CA); Joseph Arnold FISHER, Thornhill (CA); David MIKULIS, Oakville (CA); Julien POUBLANC, Toronto (CA); Behzad SHARIF, Los Angeles, CA (US); Olivia SOBCZYK, Etobicoke (CA); Kamil ULUDAG, Toronto (CA); Chau VU, Los Angeles, CA (US); John WOOD, Los Angeles, CA (US); Hsin-Jung YANG, Oakville (CA)

(72) Inventors: Adrian P. CRAWLEY, Toronto (CA); Rohan DHARMAKUMAR, Moorpark, CA (US); James DUFFIN, Toronto (CA); Joseph Arnold FISHER, Thornhill (CA); David MIKULIS, Oakville (CA); Julien POUBLANC, Toronto (CA); Behzad SHARIF, Los Angeles, CA (US); Olivia SOBCZYK, Etobicoke (CA); Kamil ULUDAG, Toronto (CA); Chau VU, Los Angeles, CA (US); John WOOD, Los Angeles, CA (US); Hsin-Jung YANG, Oakville (CA)

(21) Appl. No.: 17/789,732

(22) PCT Filed: Dec. 31, 2020
(86) PCT No.: PCT/IB2020/062602

§ 371 Date: Jun. 28, 2022

Related U.S. Application Data
(60) Provisional application No. 62/955,998, filed on Dec. 31, 2019.
(60) Provisional application No. 62/981,949, filed on Feb. 26, 2020.
(60) Provisional application No. 63/025,403, filed on May 15, 2020.

(51) Int. Cl. G01R 33/28; A61B 5/055; A61B 5/00; A61B 5/026; A61B 5/145; G01R 33/50; G01R 33/58

(52) U.S. Cl. CPC G01R 33/281; A61B 5/055; A61B 5/7289; A61B 5/7257; A61B 5/0263; A61B 5/14546; A61B 5/14542; A61B 5/0044; G01R 33/50; G01R 33/58; A61B 2560/0223

(57) ABSTRACT

Deoxyhemoglobin in a subject may be modulated to act as a contrast agent for use in magnetic resonance imaging. Sequential gas delivery may be applied to adjust the level of deoxyhemoglobin in the subject. A suitable magnetic resonance imaging (MRI) pulse sequence that is sensitive to magnetic field inhomogeneities, such as a blood-oxygen-level dependent (BOLD) sequence, may be used to detect deoxyhemoglobin as a contrast agent.

### CROSS-REFERENCE TO RELATED APPLICATION(S)

This application claims the benefit of U.S. provisional application No. 62/955,998, filed Dec. 31, 2019; U.S. provisional application No. 62/981,949, filed Feb. 26, 2020; and U.S. provisional application No. 63/025,403, filed May 15, 2020, incorporated by reference herein.

### GOVERNMENT SUPPORT

This invention was made with Government support under Grant (or Contract) No. U01HL117718 and Grant (or Contract) No. RO1HL136484, awarded by the National Institutes of Health (NIH). The Government has certain rights in this invention.

### FIELD

The present specification is directed to medical imaging of human subjects and in particular contrast agents for magnetic resonance imaging.

### BACKGROUND

Positron emission tomography (PET) using oxygen-15 is considered the gold standard for mapping blood flow metrics but it requires a cyclotron for generating a short-lived tracer. These limitations prohibit its use for generalized clinical application. Other methods for acquiring this data also suffer limitations. Single photon emission computed tomography (SPECT) uses a single pass radioactive tracer that can provide blood flow maps, but cerebral blood volume (CBV) and mean transit time (MTT) metrics are typically unavailable. Computed tomography (CT) perfusion imaging is the most widely available method for obtaining perfusion metrics but limitations include exposure to ionizing radiation, contrast reactions to the iodinated tracer, and potential tracer induced renal toxicity.

Magnetic resonance imaging (MRI) is an appealing imaging approach as it does not rely on ionizing radiation. Standard MRI perfusion imaging uses a gadolinium-based contrast agents (GBCAs) as a vascular tracer since they remain in the vasculature provided that the blood-brain-barrier is intact. Its main limitation is the non-linearity of the acquired signal versus gadolinium concentration. There is also the potential for contrast reactions and risk of nephrogenic systemic sclerosis if administered to patients with renal insufficiency.

Arterial spin labelling (ASL) MRI can measure blood flow using arterial water as an endogenous tracer but T1 relaxation decay of the tracer over time complicates quantitative measurement and ASL typically has low signal-to-noise ratio (SNR). This is not usually an issue when the vasculature is healthy, but it becomes problematic in vascular disorders when normal flow patterns are disrupted causing delay and dispersion of the labelled water. It therefore becomes difficult to determine if the signal reduction at the tissue level is secondary to delay and dispersion vs. signal loss from T1 relaxation effects or both.

### SUMMARY

It is an aspect of the present invention to provide a contrast agent for use in magnetic resonance imaging.

It is a further aspect of the present invention to provide a method of using deoxyhemoglobin in a subject as a contrast agent in magnetic resonance imaging.

It is a further aspect of the present invention that any suitable magnetic resonance imaging (MRI) pulse sequence that is sensitive to magnetic field inhomogeneities, such as a blood-oxygen-level dependent or BOLD sequence, may be used to detect deoxyhemoglobin as a contrast agent.

The above aspects can be attained by adjusting a level of deoxyhemoglobin in a subject and conducting magnetic resonance imaging on the subject using the deoxyhemoglobin of the subject as a contrast agent.

An example method includes generating a change in deoxyhemoglobin in a subject, conducting magnetic resonance imaging on the subject, and using the deoxyhemoglobin of the subject as a contrast agent for a weighted imaging of the magnetic resonance imaging.

An example method of controlling deoxyhemoglobin in a subject includes providing a gas for the subject to inhale to obtain a target lung partial pressure of oxygen and a target lung partial pressure of carbon dioxide to obtain a target level of deoxyhemoglobin in the subject's blood.

An example use of hypoventilation and/or breath holding for a subject generates deoxyhemoglobin in the subject for use as contrast agent in magnetic resonance imaging.

An example method of calibrating magnetic resonance imaging includes controlling blood deoxyhemoglobin in a subject by administering a gas that provides a lung partial pressure of oxygen and a lung partial pressure of carbon dioxide to the subject, capturing a calibrating magnetic resonance imaging signal while controlling the blood deoxyhemoglobin in the subject, obtaining a relationship of the blood deoxyhemoglobin to the calibrating magnetic resonance imaging signal, and applying the relationship to a subsequent magnetic resonance imaging signal for a tissue to obtain tissue oxygenation information.

Example devices/apparatus provide one or more processors to implement the methods, uses, and techniques discussed herein.

These together with other aspects and advantages which will be subsequently apparent, reside in the details of construction and operation as more fully hereinafter described and claimed, reference being had to the accompanying drawings forming a part hereof, wherein like numerals refer to like parts throughout.

### BRIEF DESCRIPTION OF THE DRAWINGS

FIG. 1 is a block diagram of a system to calibrate magnetic resonance imaging.

FIG. 2 is a flowchart of a method of calibrating magnetic resonance imaging.

FIG. 3 is a graph of P_{ET}O₂ and SₐO₂ according to one example.

FIG. 3A illustrates a color copy of FIG. 3.

FIG. 4 is a set of graphs of the BOLD signal according to one example.

FIG. 5 is a set of graphs of the MRI signal according to one example.

FIG. 6 is a graph of the BOLD signal according to one example.

FIG. 6A illustrates a color copy of FIG. 6.

FIG. 7 is a brain map of the percent change in BOLD signal according to one example.

FIG. 7A is a color copy of FIG. 7.

FIG. 8 is a graph of the contrast-to-noise ratio according to one example.

FIG. 8A is a color copy of FIG. 8.

FIG. 9 is a brain map of the cerebral blood volume according to one example.

FIG. 9A is a color copy of FIG. 9.

FIG. 10 is a brain map of the cerebral blood flow according to one example.

FIG. 10A is a color copy of FIG. 10.

FIG. 11 is a brain map of the mean transit time according to one example.

FIG. 11A is a color copy of FIG. 11.

FIG. 12 is a graph of the BOLD signal according to one example.

FIG. 12A is a color copy of FIG. 12.

FIG. 13 is an annotated schematic diagram of example of modelling shunting according to one example.

FIG. 14 is an annotated schematic diagram of generalized bidirectional shunting.

FIG. 15 is a graph of an example sinusoidal pO₂ stimulus oscillating between 30 and 80 Torr.

FIG. 16 is a graph of a predicted arterial saturation for the stimulus of FIG. 15.

FIG. 17 is a plot of an observed change in BOLD signal intensity for the stimulus of FIG. 15.

FIG. 18 is a graph of an estimated pO₂ waveform necessary to produce sinusoidal fluctuations in oxygen saturation between 60% and 95%, in which the “True” waveform represents a model prediction and the “Approximate” curve is an approximation using ramps and half-sinusoids that are programmable on a sequential gas delivery device, such as the device of FIG. 1.

FIG. 19 is a plot of a global BOLD signal from a paradigm of four consecutive waveforms.

FIG. 20 are images of cerebral blood flow (CBF) and cerebral blood volume (CBV) maps derived from a normal volunteer using the pO₂ waveform of FIG. 18.

FIG. 20A is a color copy of FIG. 20.

FIG. 21 are images of CBF and CBV maps from the same subject as in FIG. 20, assuming that blood had a quadratic relationship with oxygen saturation and tissue varied with an exponent of 1.3

FIG. 21A is a color copy of FIG. 21.

### DETAILED DESCRIPTION

Embodiments and examples of the present invention will be described with respect to the use of deoxyhemoglobin as a contrast agent in magnetic resonance imaging and a method for using deoxyhemoglobin in a subject as a contrast agent in magnetic resonance imaging.

“Blood-oxygen-level dependent imaging” or “BOLD imaging” herein refers to an MRI technique for detecting deoxygenated hemoglobin and oxygenated hemoglobin in a subject. Deoxygenated hemoglobin is paramagnetic whereas oxygenated hemoglobin is not, and therefore the former will cause local dephasing of protons, and thus reduce the returned signal from the tissues in the immediate vicinity. In BOLD, T2* weighted sequences are used to detect this change.

“Bolus” herein refers to a discrete amount of a test substance that is rapidly delivered to a subject to hasten or magnify a physiological response.

“Cardiac output” or “\dot{Q}” herein refers to the volume of blood pumped by the heart per unit time, usually expressed in liters per minute (L/min).

“Contrast agent” herein refers to a test substance administered to a subject used to increase the contrast of structures or fluids within the body in MRI. Contrast agents absorb or alter electromagnetism emitted by the MRI device.

“Deoxyhemoglobin” or “dOHb” herein refers to hemoglobin molecules that are unsaturated by oxygen.

“Deoxyhemoglobin concentration” or “[dOHb]” herein refers to the concentration of deoxyhemoglobin in blood. Usually expressed in grams per deciliter (g/dL).

“Hypoxic gas” herein refers to a gas having a partial pressure of oxygen (PO₂) that, when inhaled, would leave hemoglobin in the lung partially unsaturated with oxygen. The PO₂ of a hypoxic gas is typically less than 150 mmHg.

“Left ventricular end-diastolic volume” or “LVEDV” refers to the volume of blood in the left ventricle at the end diastole, immediately preceding contraction.

“Repetition time” or “TR” herein refers to a repeat time of radiofrequency change in a magnetic direction of protons to initiate a decay of proton orientation, or the time interval between repeat BOLD signals.

“Shunt fraction” or “SF” herein refers to the fraction of potential systemic flow crossing to pulmonary flow and vice versa.

“Stroke volume” or “SV” herein refers to the volume of blood ejected from the heart during contraction.

“T2” herein refers to a time constant for the decay of transverse magnetization during the MRI process.

“T2*” herein in refers to the observed or effective T2 value in the MRI process.

Hemoglobin is contained in red blood cells and thus is entirely intravascular. Blood returns from the tissues to the heart and pulmonary arteries with a PO₂ of about 40 mmHg and arterial hemoglobin saturation (SₐO₂) of about 70%. In healthy people breathing room air at sea level, the inspired PO2 is about 150 mmHg, and about 110 mmHg in the lung alveoli. During its transit through the alveolar capillaries, inhaled O₂ diffuses into the red blood cells raising the SₐO₂%. The relationship between the arterial PO₂ (PₐO₂) and SₐO₂ is sigmoidal with Hb being fully saturated at PO₂ of about 100 mmHg (Balaban et al., 2013). At PO₂ in the lung of less than 100 mmHg, some Hb remains unsaturated, termed deoxyhemoglobin (dOHb). For example, if the gas in the alveoli had a PO₂ of 40 mmHg, the blood from the pulmonary artery would pass into the pulmonary vein, and out into the arterial tree unchanged, with a PO₂ of 40 and SₐO₂ of 70%. By targeting the PO₂ in the lung, the PO₂ in the blood returning to the heart and out into the arterial tree will have the same PO₂, and thus the corresponding SₐO₂.

Oxygenated Hb (OHb) is diamagnetic, and does not affect T2* relaxation. Deoxygenated hemoglobin (dOHb) is paramagnetic and reduces T2* signal in proportion to its concentration, the so called Blood Oxygen Level Dependent (BOLD) effect. If rapid changes in SₐO₂ can be implemented in the lung, the consequent change in suceptibility can be followed out into the tissues, thus acting as a contrast agent.

The ideal application of the contrast would be a square wave change in susceptibility, as would occur in the instantaneous injection of contrast agent into an artery. This is hard to implement via the lung. An abrupt reduction of the O₂ concentration in inspired air requires many breaths before the lung comes to a new steady state PO₂. An abrupt increase in PO₂ in inspired air requires the same number of breaths; but raising the lung PO₂ above the PO₂ of 100 mmHg does not change the dOHb concentration ([dOHb]) and thus the susceptibility. The third confounding element is that any change in breathing pattern would change both the PO₂ (i.e., the baseline condition), as well as the partial pressure of carbon dioxide in the arterial blood (PaCO₂) which in turn affects cerebral blood flow and BOLD signal. Furthermore, the partial pressure of carbon dioxide (PCO₂) has an adjuvant effect on the association and dissociation of O₂ with hemoglobin. Increased PCO₂ tends to push O₂ off the hemoglobin, increasing the [dOHb], and vice versa. This is termed the Haldane effect.

Therefore, to meet the requirements for use of [dOHb] as a contrast agent for measuring organ tissue perfusion, four conditions must be met: (1) maintain baseline blood PO₂ levels; in this case, at or below 100 mmHg. (2) induction of an abrupt change in [dOHb] by inducing an abrupt change PO₂ in the lung between one breath and the next; (3) maintain isocapnia independent of changes in ventilation to prevent CO₂-effected changes in blood flow; and finally, (4) identify the relationship between BOLD signal and [dOHb]. Implementing the abrupt change in arterial [dOHb] in the lung would allow it to arrive at target organs with minimal dispersion, approximating plug flow. This results in large, abrupt, targetable (i.e. repeatable) changes in [dOHb]. Rather than non-linear wash-out kinetics seen with increases in flow, in the case of changes in [dOHb], the flow remains unchanged and the BOLD signal in the microcirculation would have a highly linear relationship with the PaO₂ (Uludag et al., 2009)

The PaO₂ of the subject may be controlled with a sequential gas delivery (SGD) device.

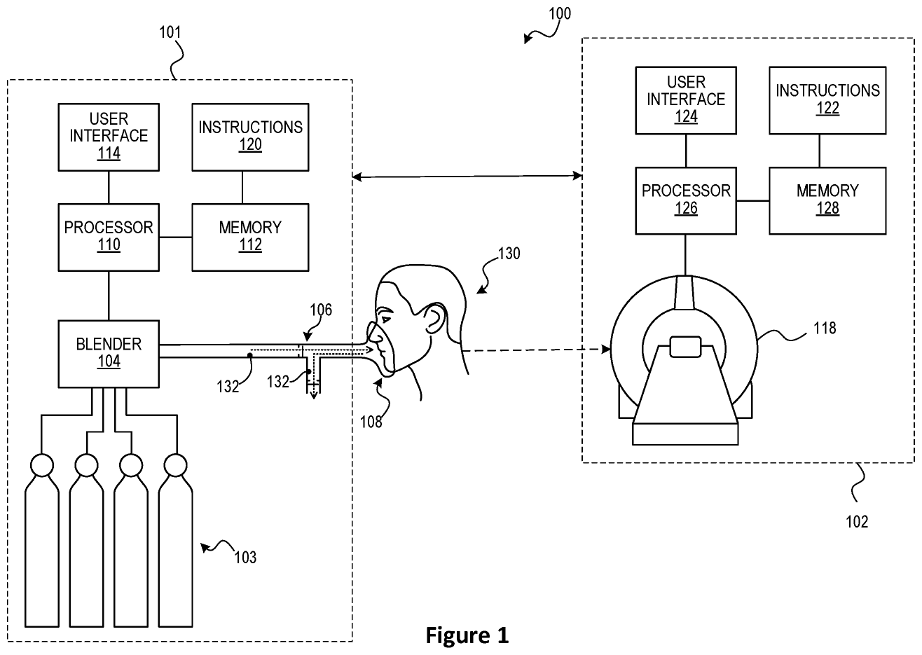

FIG. 1 shows a system 100 for using dOHb as a contrast agent. The system 100 includes an SGD device 101 to provide sequential gas delivery to a subject 130 and target a PₐO₂ while maintaining normocapnia. The system 100 further includes a magnetic resonance imaging (MRI) system 102. The device 101 includes gas supplies 103, a gas blender 104, a mask 108, a processor 110, memory 112, and a user interface device 114. The device 101 may be configured to control end-tidal PCO₂ and end-tidal PO₂ by generating predictions of gas flows to actuate target end-tidal values. The device 101 may be an RespirAct™ device, made by Thornhill Medical™ of Toronto, Canada, specifically configured to implement the techniques discussed herein. For further information regarding sequential gas delivery, U.S. Pat. No. 8,844,528, US Pub. 2018/0043117, and U.S. Pat. No. 10,850,052, which are incorporated herein by reference, may be consulted.

The gas supplies 103 may provide carbon dioxide, oxygen, nitrogen, and air, for example, at controllable rates, as defined by the processor 110. The following gas mixtures may be used. Gas A: 10% O₂, 90% N₂; Gas B: 10% O₂, 90% CO₂; Gas C: 100% O₂; and a calibration gas: 10% O₂, 9% CO₂, 81% N₂.

The gas blender 104 is connected to the gas supplies 102, receives gasses from the gas supplies 102, and blends received gasses as controlled by the processor 110 to obtain a gas mixture, such as a first gas (G1) and a second gas (G2) for sequential gas delivery.

The second gas (G2) is a neutral gas in the sense that it has about the same PCO₂ as the gas exhaled by the subject 130, which includes about 4% to 5% carbon dioxide. In some examples, the second gas (G2) may include gas actually exhaled by the subject 130. The first gas (G1) has a composition of oxygen that is equal to the target P_{ET}O₂ and preferably no significant amount of carbon dioxide. For example, the first gas (G1) may be air (which typically has about 0.04% carbon dioxide), may consist of 21% oxygen and 79% nitrogen, or may be a gas of similar composition, preferably without any appreciable CO₂.

The processor 110 may control the blender 104, such as by electronic valves, to deliver the gas mixture in a controlled manner.

The mask 108 is connected to the gas blender 104 and delivers gas to the subject 130. A valve arrangement 106 may be provided to the device 101 to limit the subject's inhalation to gas provided by the blender 104 and limit exhalation to the room. An example valve arrangement 106 includes an inspiratory one-way valve from the blender 104 to the mask 108, a branch between the inspiratory one-way valve and the mask 108, and an expiratory one-way valve at the branch. Hence, the subject 130 inhales gas from the blender 104 and exhales gas to the room.

The gas supplies 102, gas blender 104, and mask 108 may be physically connectable by conduits, such as tubing, to convey gas. Any number of sensors 132 may be positioned at the gas blender 104, mask 108, and/or conduits to sense gas flow rate, pressure, temperature, and/or similar properties and provide this information to the processor 110. Gas properties may be sensed at any suitable location, so as to measure the properties of gas inhaled and/or exhaled by the subject 130.

The processor 110 may include a central processing unit (CPU), a microcontroller, a microprocessor, a processing core, a field-programmable gate array (FPGA), an application-specific integrated circuit (ASIC), or a similar device capable of executing instructions. The processor may be connected to and cooperate with the memory 112 that stores instructions and data.

The memory 112 includes a non-transitory machine-readable medium, such as an electronic, magnetic, optical, or other physical storage device that encodes the instructions. The medium may include, for example, random access memory (RAM), read-only memory (ROM), electrically erasable programmable read-only memory (EEPROM), flash memory, a storage drive, an optical device, or similar.

The user interface device 114 may include a display device, touchscreen, keyboard, buttons, and/or similar to allow for operator input/output.

Instructions 120 may be provided to carry out the functionality and methods described herein. The instructions 120 may be directly executed, such as a binary file, and/or may include interpretable code, bytecode, source code, or similar instructions that may undergo additional processing to be executed.

The instructions 120 prospectively target an end tidal partial pressure of oxygen (P_{ET}O₂) by controlling the SGD device 101 to deliver a first volume of a first gas (G1) to the subject 130 over a first portion of an inspiration by the subject 130. The first volume is selected to be less than or equal to an estimated or expected alveolar volume (VA) of the subject 130 when the subject is breathing normally. The first gas (G1) has a PO₂ that is equal to the targeted P_{ET}O₂ and preferably no significant amount of CO₂. The instructions 120 deliver a second volume of a second, neutral gas (G2) to the subject 130 over a second portion of the inspiration. The second gas is a neutral gas that has a PCO₂ corresponding to the PCO₂ in the exhaled gas and preferably the same amount of CO₂ as present in the previously exhaled breath. The second gas (G2) is unlimited in the sense that during normal or deep breathing, the end of the inspiration will contain as much second gas (G2) as needed.

The instructions 120 measure a PO₂ at the end of an exhalation by the subject 130 occurring after delivery of the first and second gases (G1, G2), that is the P_{ET}O₂ while the subject breaths.

The instructions 120 may target a first P_{ET}O₂ over a first period of time and a second P_{ET}O₂ over a second period of time. The first targeted P_{ET}O₂ is selected to induce hypoxia in the patient. In some examples, the first targeted P_{ET}O₂ is approximately 40 mmHg. In some examples, the first targeted P_{ET}O₂ is approximately 50 mmHg. In some examples, the first targeted P_{ET}O₂ is approximately 60 mmHg. In some examples, the first targeted P_{ET}O₂ is approximately 70 mmHg. In some examples, the first targeted P_{ET}O₂ is approximately 80 mmHg. The second targeted P_{ET}O₂ is a value greater than the first targeted P_{ET}O₂. In some examples, the second targeted P_{ET}O₂ is approximately 60 mmHg. In some examples, the second targeted P_{ET}O₂ is approximately 70 mmHg. In some examples, the second targeted P_{ET}O₂ is approximately 80 mmHg. In some examples, the second targeted P_{ET}O₂ is approximately 90 mmHg. In some examples, the second targeted P_{ET}O₂ is approximately 100 mmHg. In some examples, the second targeted P_{ET}O₂ is approximately 110 mmHg. In some examples, the second targeted P_{ET}O₂ is approximately 120 mmHg. In some examples, the second targeted P_{ET}O₂ is approximately 130 mmHg. In some examples, the second targeted P_{ET}O₂ is approximately 140 mmHg.

The instructions 120 may measure the first and second periods of time in breaths by the subject or in seconds and minutes. In some examples, the subject can be directed to breathe at a frequency of, for example, 30 beats per minute. In these examples, the duration of the second period of time can be 2 seconds, 4 seconds, 6 seconds, or any suitable multiple of 2. Durations and granularity will vary with breathing frequency. The breathing rate of the subject may be controlled and changes to, so as to individualize the granularity and precision of durations of stimulus and baseline. The first or second periods of time may be less than 3 minutes to reduce the effect of hypoxia on blood flow. Since blood flow increases approximately 3 minutes after the onset of hypoxia, the instructions 120 may be programmed to return the subject 130 to normoxia within 3 minutes.

The instructions 120 may target a first and second P_{ET}O₂ in any order. In some examples, the instructions 120 may target the second P_{ET}O₂ and then target the first P_{ET}O₂. In other examples, the instructions 120 may target the first P_{ET}O₂ and then target the second P_{ET}O₂. In further implementations, the instructions 120 may alternate between targeting the first and second P_{ET}O₂. In one implementation, the instructions 120 target the first P_{ET}O₂ for 90 seconds, target the second P_{ET}O₂ for 15 seconds, target the first P_{ET}O₂ for a further 90 seconds, and target the second P_{ET}O₂ for a further 15 seconds. This may be repeated any number of suitable times. In some implementations, the instructions 120 may start and end with targeting the first P_{ET}O₂.

The instructions 120 apply Equation 1 or equivalent to compute the SₐO₂ using the P_{ET}O₂ measured by the device 101. The dissociation constant (K) and the Hill coefficient (n) are determined using methods described in Balaban et al., 2013.

$${S}_{a} {O}_{2} = 100 \frac{{K ( {P}_{ET} {O}_{2} )}^{n}}{1 + {K ( {P}_{ET} {O}_{2} )}^{n}} \qquad Equation 1$$

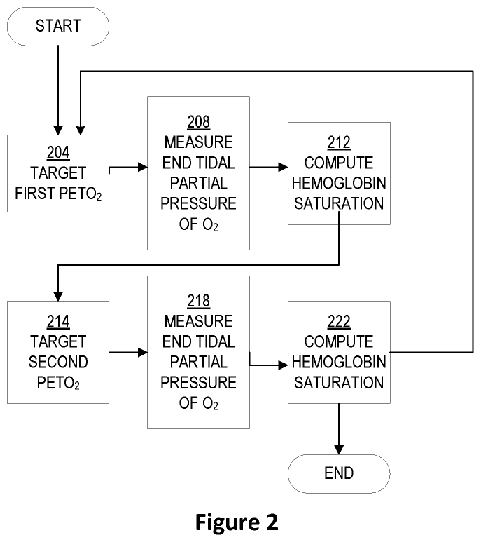

FIG. 2 shows an example method 200 of generating a deoxyhemoglobin bolus in a subject using SGD. The method 200 may be implemented by instructions 120. At block 204, the instructions 130 control the device 101 to target the first P_{ET}O₂ corresponding to hypoxia in the subject. The device 101 targets the first P_{ET}O₂ for a first period of time. During this first period, the instructions 120 control the device 101 to measure the P_{ET}O₂ at block 208. Using Equation 1 and the measured P_{ET}O₂, the instructions compute the SₐO₂ at block 212. At block 214, the instructions 120 control the device 101 to target a second P_{ET}O₂ for a second period of time. During this second period, the instructions 120 control the device 101 to measure the P_{ET}O₂ at block 218. Using Equation 1 and the measured P_{ET}O₂, the instructions 120 compute the SₐO₂. From block 222, the method 200 may return to block 204 and repeat the subsequent steps. The method may be repeated any suitable number of times.

As explained above with regard to FIG. 1, blocks 214, 218, and 222 could be performed before blocks 204, 208, and 212. In other words, some implementations target the second P_{ET}O₂ and then target the first P_{ET}O₂.

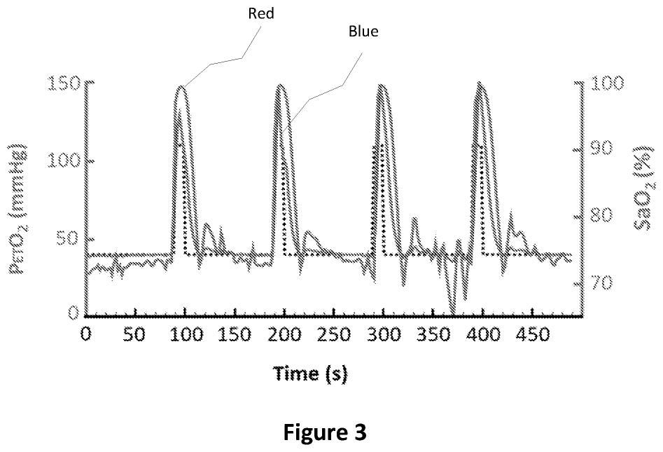

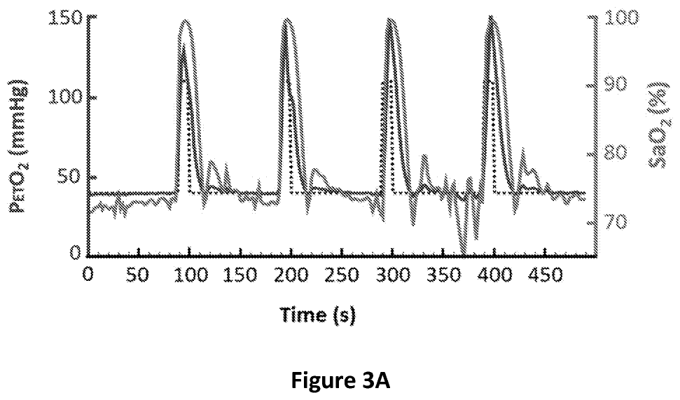

FIG. 3 is a graph showing the change in P_{ET}O₂ and SₐO₂ in a subject during the implementation of the method 200 described in FIG. 2. In this implementation, the method 200 starts at block 204 by implementing the first targeted P_{ET}O₂ and returns to block 204 three times. The dotted line shows the targeted P_{ET}O₂, the blue line shows the P_{ET}O₂ measured by the device 101 and the red line shows the calculated SₐO₂.

As the instructions 120 are implemented by the device 101, the MRI system 102 conducts magnetic resonance imaging on the subject 130. A suitable MRI system may include an imaging device such as a 3T MRI system (Signa HDxt—GE Healthcare, Milwaukee). The MRI system 102 may further include a processor 126, memory 128, and a user interface 124. In some implementations, the MRI system 102 and the SGD device share a common memory, process, user interface, and instructions. However, in the present disclosure, the MRI system 102 and the SGD device 101 will be described as having respective processors, user interfaces, memories, and instructions. The processor 110 of the SGD device 101 transmits data to the processor 126 of the MRI system 102. The system 100 may be configured to synchronize MRI imaging obtained by the MRI system 102 with measurements obtained by the SGD device 101.

The processor 126 may retrieve operating instructions 122 from the memory or may receive operating instructions 122 from the user interface 124. The operating instructions 122 may include image acquisition parameters. The parameters may include an interleaved echo-planar acquisition consisting of a number of contiguous slices, a defined isotropic resolution, a diameter for the field of view, a repetition time, and an echo time. In one implementation, the number of contiguous slices is 27, the isotropic resolution is 3 mm, the field of view is 19.6 cm, the echo time is 30 ms, and the repetition time (TR) is 2000 ms, however a range of values will be apparent to a person of ordinary skill in the art. The operating instructions 122 may also include parameters for a high-resolution T1-weighted SPGR (Spoiled Gradient Recalled) sequence for co-registering the BOLD images and localizing the arterial and venous components. The SPGR parameters may include a number of slices, a dimension for the partitions, an in-plane voxel size, a diameter for the field of view, an echo time, and a repetition time. In one implementation, the number of slices is 176 m, the partitions are 1 mm thick, the in-plane voxel size is 0.85 by 0.85 mm, the field of view is 22 cm, the echo time is 3.06 ms, and the repetition time is 7.88 ms.

The images acquired by the MRI system are stored in memory and analyzed by the processor 126. The processor 126 may be configured to analyze the images using image analysis software such as Matlab 2015a and AFNI (Cox, 1996) or other processes generally known in the art. As part of the analysis, the processor may be configured to perform slice time correction for alignment to the same temporal origin and volume spatial re-registration to correct for head motion during acquisition. The processor may be further configured to perform standard polynomial detrending. In one implementation, the processor 126 is configured to detrend using AFNI software 3dDeconvolve to obtain detrended data (labelled as Sₜ). The baseline BOLD signal (S₀) can then be defined as the mean of the BOLD signal (Sₜ) over one or more intervals. Those intervals can include portions of the first periods of time, selected by the processor 126 to omit sections of time immediately following the second periods of time when the signal might not have fully returned to a stable baseline. In an implementation where the first period of time is 90 seconds, the second period of time is 15 seconds, and the method 200 is repeated three times, the intervals include 90 seconds before the first second period, and 10 seconds before each subsequent second period. If blocks 204 to 212 are repeated immediately before the end of the method 200, the intervals can also include the 10 seconds before the end of the method 200.

The processor 126 is further configured to calculate the scaled BOLD signal (S_{c,t}) using Equation 2

$${S}_{c , t} = \frac{{S}_{t}}{{S}_{0}} - 1 \qquad Equation 2$$

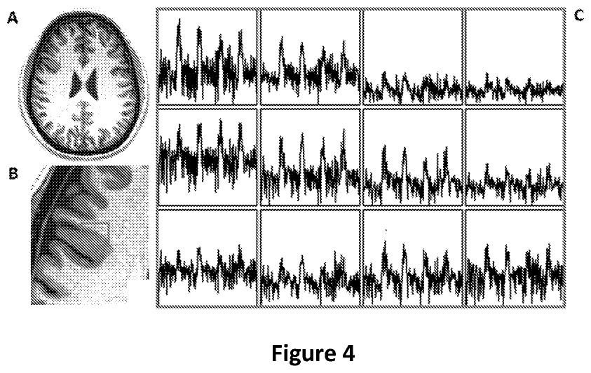

FIG. 4 shows an example of the BOLD signal calculated for 12 voxels. Panel A shows the location of the 12 voxels, Panel B shows location details of the 12 voxels which include both gray and white matter, and Panel C shows the scaled BOLD signal from each of the 12 voxels outlined in A and B. Note that the voxels containing primarily white matter correspond to a reduced scaled BOLD signal as compared with the voxels containing primarily gray matter due to the reduced vascularity in white matter.

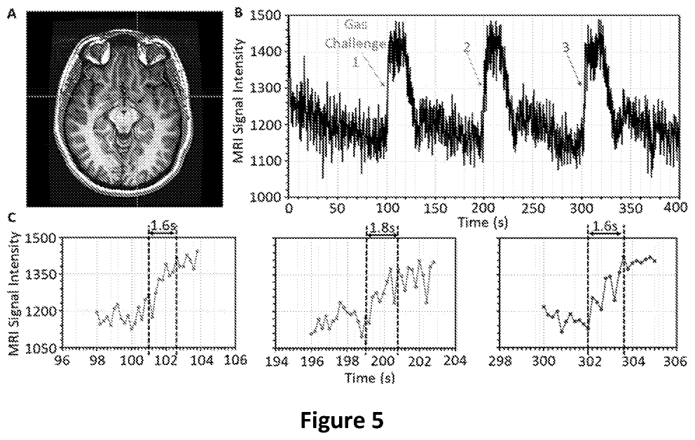

FIG. 5 shows the BOLD signal for a voxel overlying the middle cerebral artery of a subject. In this example, the repetition time was 200 ms and the method 200 was repeated twice. Panel A shows the location of the voxel in a subject's brain. Panel B shows the BOLD signal against time. Panel C shows details of the BOLD signal during intervals after the device 101 begins targeting the second P_{ET}O₂ (these intervals will subsequently be referred to as “gas challenges”). During the gas challenges, the subject's brain is responding to the change from hypoxia to normoxia.

The processor 126 may be further configured to calculate a time delay (TD) map using cross-correlation between S_{c,t} and multiple SₐO₂ curves that are time-shifted. The processor 126 may be configured to time shift the curve by a suitable duration of time, for example from 0 to 5 seconds by intervals of 0.1 seconds. For each time shift, the processor 126 may be configured to compute a correlation (R) between the SₐO₂ and S_{c,t} and select the time shift for each voxel that maximizes the R.

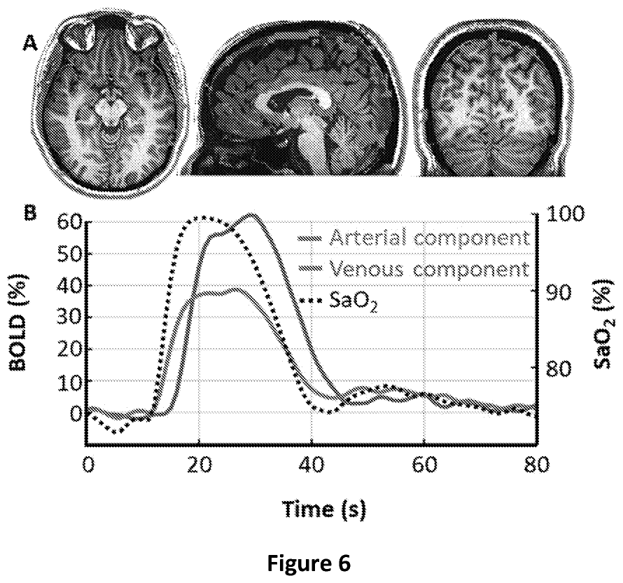

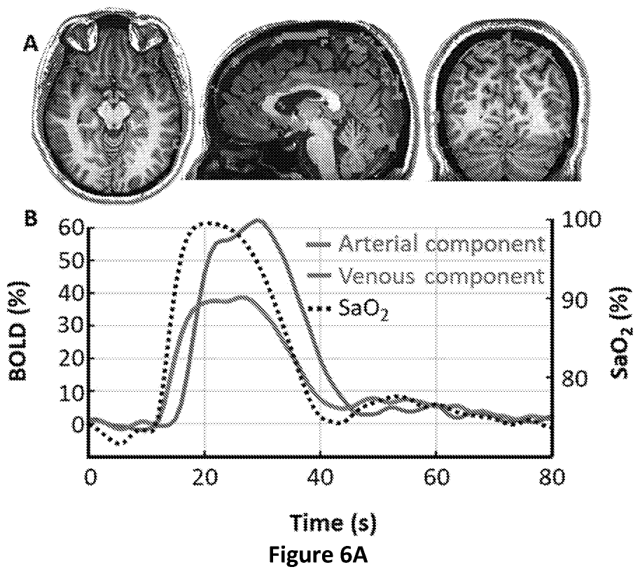

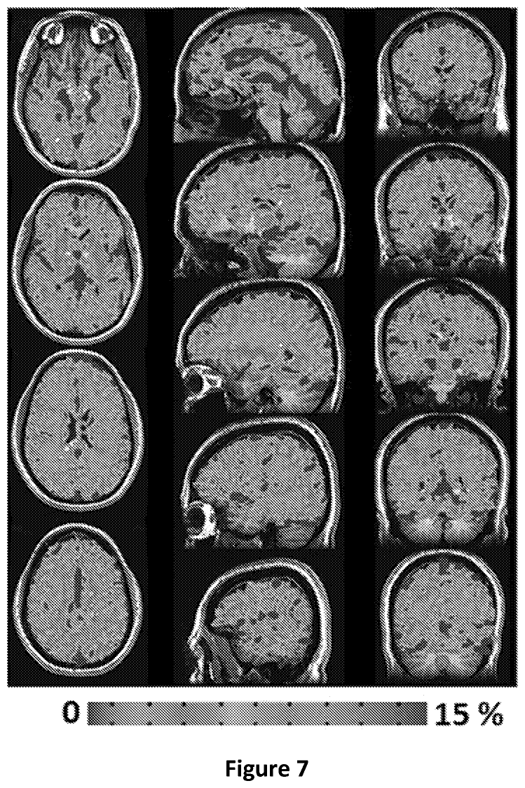

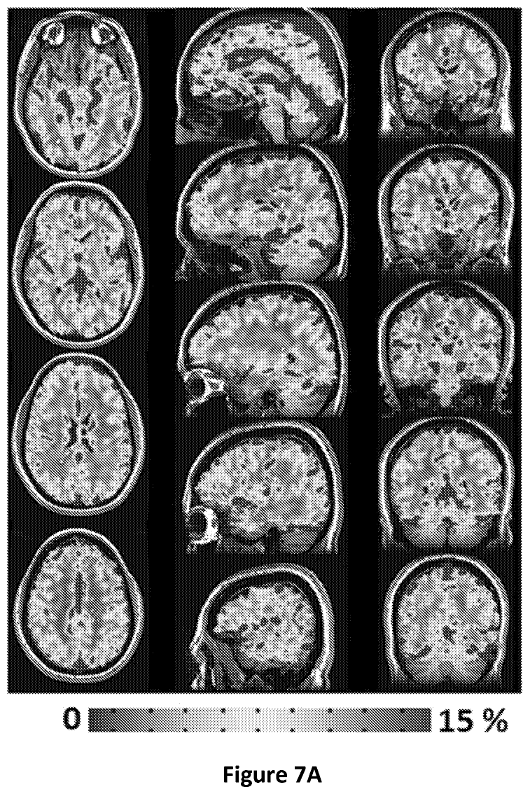

The processor 126 may be further configured to predict the locations of arterial and venous structures based on the percent change in the BOLD signal (ΔS), R and time delay. The method for computing ΔS is described below with respect to FIG. 7. At panel A, FIG. 6 shows images of a subject's brain including predicted locations of arterial and venous structures based on ΔS, R, and time delay. In this example, arterial voxels were predicted to have a ΔS greater than 20 percent, an R value greater than 0.8, and a time delay less than 1.5 seconds. Also in this example, venous voxels were predicted to have a ΔS greater than 20 percent, an R value greater than 0.8, and a time delay greater than 3 seconds. Panel B shows a graph of the BOLD signal over 4 repetitions of the method 200 for arterial voxels (red) and venous voxels (blue). The graph in panel B also shows an oxygen saturation curve (dotted line) which was measured by the device 101 at the subject's mouth but time shifted to correspond to the change from baseline in the arterial curve. FIG. 6 shows that the amplitude of BOLD signal for arterial voxels is less than the amplitude of venous voxels because arteries have smaller diameters and typically do not fill an entire voxel. In contrast, veins have larger diameters which may entirely contain one or more voxels.

ΔS can be calculated by and mapped to the images obtained from the MRI system. First, S_{c,t} is regressed against a voxel-wise shifted SₐO_{2,t}^{shifted} to calculate the slop of regression according to Equation 3, where α is the slope of the regression and εₜ are the residuals.

S_{c,t}=α·SₐO_{2,t}^{shifted}+εₜ Equation 3

Next, ΔS can be calculated according to Equation 4.

ΔS=α·(max(SₐO_{2,t}^{shifted})−min(SₐO_{2,t}^{shifted})) Equation 4

FIG. 7 shows a map of ΔS expressed in percentages. In these images, the arteries and veins of the subject's brain are clearly delineated from the other brain structures with changes under 15 percent.

The contrast to noise ratio (CNR) can be assessed using ΔS and the residuals Et according to Equation 5.

$$CNR = \frac{Δ S}{std ( {ε}_{t} )} \qquad Equation 5$$

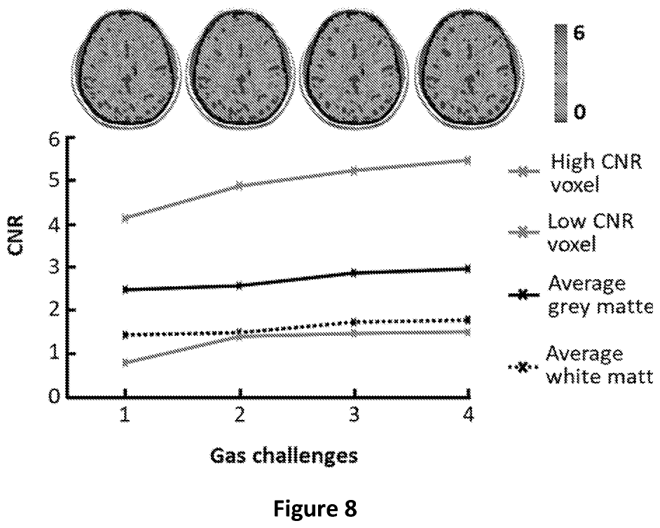

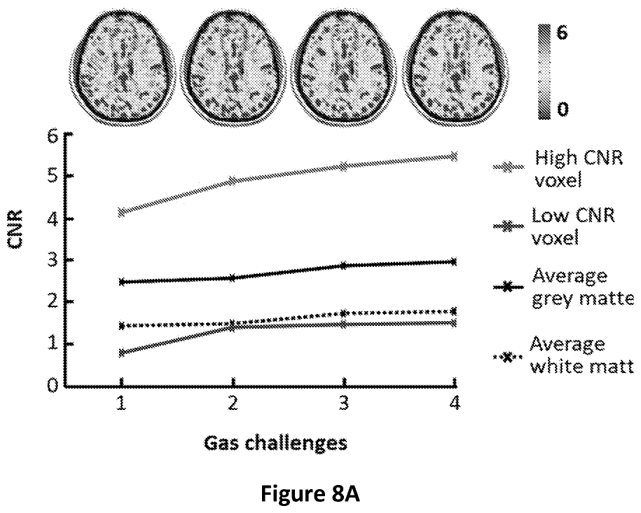

CNR can be calculated for each second time period by truncating the time series. FIG. 8 shows an example where the CNR is calculated over the course of four gas challenges. In this example, the CNR is averaged for high CNR voxels, low CNR voxels, voxels primarily containing gray matter, and voxels primarily containing white matter. FIG. 8 shows there is an improvement in average CNR from the first gas challenge to the second gas challenge. However, the third and fourth gas challenges provide little improvement in average CNR. The average CNR is generally similar for the second, third, and fourth gas challenges.

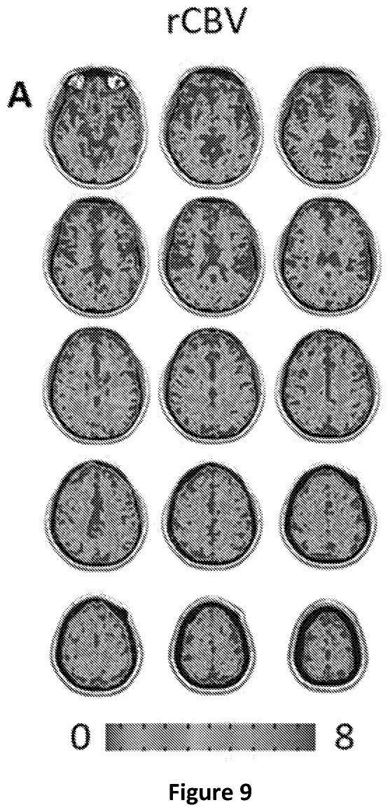

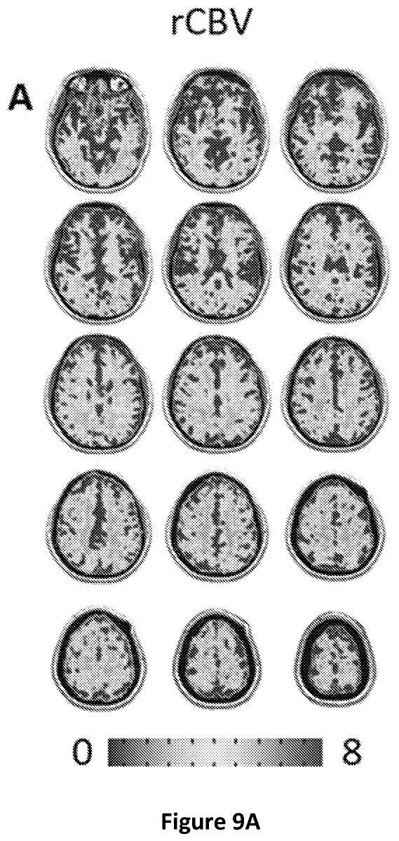

The processor 126 is further configured to calculate CBV in a subject. FIG. 9 shows a map of the CBV values calculated according to the following calculations. The relative cerebral blood volume (CBV) is assumed to be approximately equal to the area under the curve (AUC), which is defined as the area between the S_{c,t} curve and the baseline (S_{c,0}=1). AUC can be calculated according to Equation 6.

$$AUC = \frac{Σ ( {S}_{c , t} - 1 ) + Σ \vert ( {S}_{c , t} - 1 ) \vert}{2} \qquad Equation 6$$

The processor 126 is further configured to calculate the quantitative CBV map according to Equation 7. In Equation 7, ρ represents tissue density and is estimated to be 1.04 g/cc, and k_{H} represents the difference in hematocrit in large vessels and capillaries and is estimated to be 0.73.

$$CBV [ \frac{mL}{g} ] = ( \frac{{k}_{H}}{ρ} ) \cdot ( \frac{AUC}{{AUC}_{v e n o u s}} ) \qquad Equation 7$$

Standard tracer kinetic modeling can be used to calculate mean transit time (MTT) and cerebral blood flow (CBF) according to Equation 8:

Sₜ=AIFₜ└(CBF×Rₜ) Equation 8

In Equation 8, Rₜ is the residue function, ⊗ denotes the convolution operator and AIFₜ represents the arterial input function. Prior art methods of obtaining true quantitative values strongly depends on determining the shape and size of the AIF. For dynamic susceptibility contrast imaging, where a contrast agent is injected intravenously, it is greatly dispersed on arrival at tissues so the arterial input function (AIF) needs to be determined prior to calculating tissue perfusion profiles. These methods are complex and inexact. One advantage of the present disclosure is that the change in susceptibility in the way of change in [dOHb] occurs abruptly and uniformly in all blood passing the pulmonary capillaries. In a healthy lung, the only major source of dispersion is in the left ventricle. FIG. 4 shows that there is indeed little dispersion with a time constant of washout from the left ventricle of about 1.5 second (implying an ejection fraction of 67%, see discussion below). In that case, assuming a square wave arterial input function (AIF) introduces much less error than measuring a widely dispersed arterial input function (AIF) from an intravenous injection of contrast agent. SₐO₂ is scaled such that AUCₐᵣₜₑᵣᵢₐₗ is the same as AUCᵥₑₙₒᵤₛ. To solve the convolution, the residue function can be a pre-defined function of unknown parameters. In some examples, the residue function is defined as an exponential with time constant MTT as shown in Equation 9:

Rₜ=e^{-t/MTT} Equation 9

This function is equal to 1 at time 0 and was set to 0 at time equal to 5×MTT.

With those specific settings, the kinetic model is re-written in Equation 10:

S_{c,t}=AIFₜ⊗(CBF×e^{-t/MTT}) Equation 10

Or, using the calculated changes in SaO₂ as the arterial input function (AIF):

S_{c,t}=SaO2⊗(CBF×e^{-t/MTT}) Equation 11

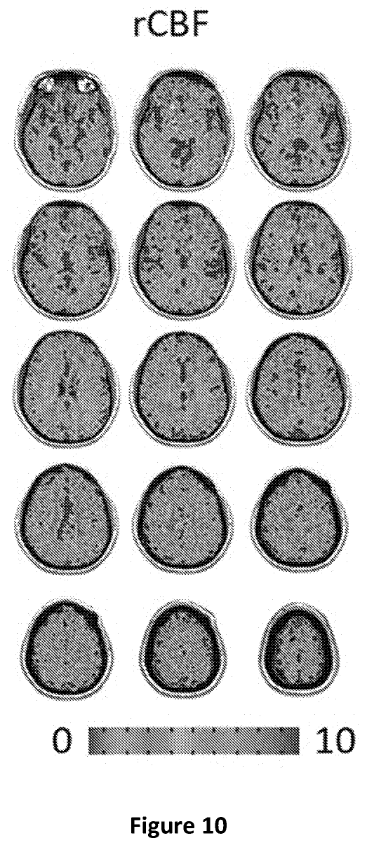

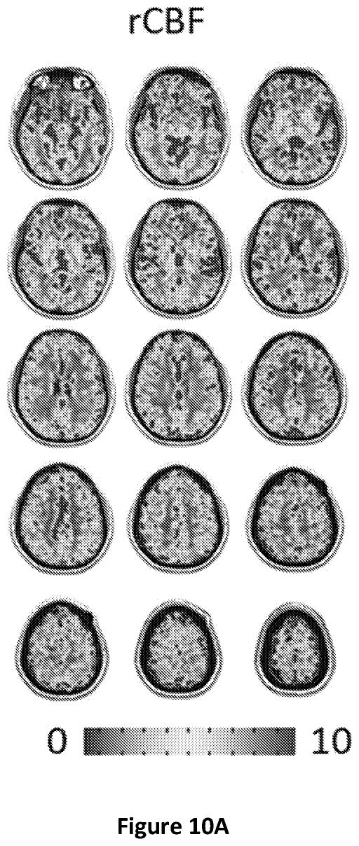

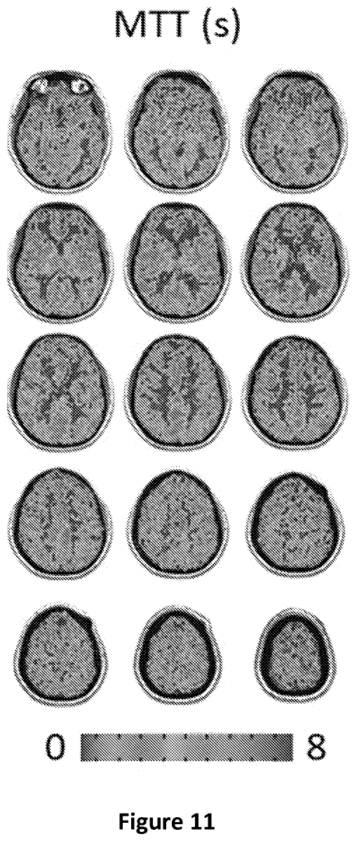

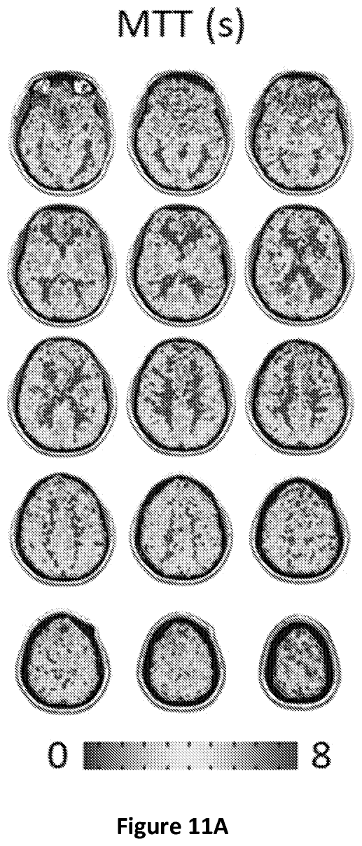

The processor 126 can the calculate the unknown parameters CBF and MTT using a least square fitting procedure. More specifically, multiple residue functions of variable MTT rnaging from o to 12 seconds were generated using 0.2 second temporal resolution and convolved with the AIFₜ. S_{c,t} is then linearly regressed against each of those functions (AIFₜ⊗e^{-t/MTT} or SaO2⊗e^{-t/MTT}). The regression with the best correlation to S_{c,t} corresponds to MTT and its slope is equal to CBF. Maps of the perfusion metrics (MTT and rCBF) can be seen in FIGS. 10 and 11. FIG. 10 shows an example of a brain map of rCBF in a subject. Figure shows an example of a brain map of MTT in a subject. Note the close mode of calculation of each is reflected in the reciprocal nature of the images, where long MTT is reflected in low rCBF as it should be based on the Central Volume Principle where CBF=CBV/MTT

During the implementation of the method 200, the inspired gas enters all alveoli substantially simultaneously. Therefore, the change in [dOHb] in the lug takes places substantially simultaneously in all alveolar capillaries, and the [dOHb] in the pulmonary vein undergoes a step change and proceeds a plug flow. When the leading edge of this new cohort of blood enters the heart, it must “wash out” the residual blood from the left ventricle (LV). This is the major cause of dispersion of the assumed square wave change of [dOHb] entering the heart. Assuming there is a linear relationship between BOLD and [dOHb] then the time constant (τ) of the change in BOLD is equal to the time constant (τ) of the change of [dOHb].

The normal healthy adult male left ventricular end-diastolic volume (LVEDV) is nominally 120 ml. The exchanges of blood in the left ventricle occurs during each heartbeat. In this case the subject was a healthy adult male. The heart rate was about 60 beats per minute (bpm). FIG. 8 shows the calculation of a time constant (r) in this example, assuming the transition is a first order exponential is about 1.5 s. This means that the 120 ml LVEDV is replaced once by 1.5 heart beats, or stroke volume of 80 ml. This predicts an LVEF=80 ml/120 ml=67%, a normal nominal value. For a heart rate of 60, the cardiac output is calculated at about 5 L/min, the stated normal nominal value.

The system 100 may be further used to measure shunt volume (SV), left ventricular end-diastolic volume (LVEDV), left ventricular ejection fraction (LVEF) and cardiac output (\dot{Q}) of the subject 130.

First, the SGD device 101 implements a change in PO₂ in the alveoli changing the [dOHb] in a single breath. The MRI system 102 monitors the BOLD signal in a selected artery, which is translated to the arterial input function (AIF). The instructions 122 impose a TR of less than 2000 ms. Ideally the instructions 122 impose a TR of 200 ms or shorter. The BOLD acquisition can also be synchronized to the cardiac cycle using cardiac gating with an electrocardiogram or plethysmography to yield TR values ranging from around 500 to 1200 ms. The device 101 is further configured to measure the heart rate of the subject 130 why the MRI is monitoring the BOLD signal.

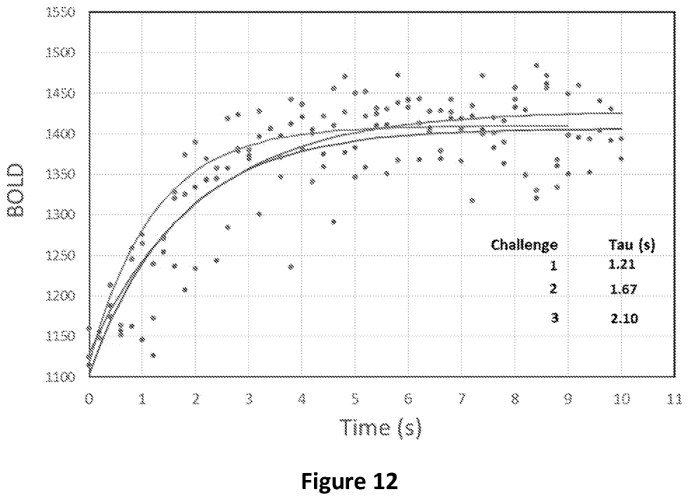

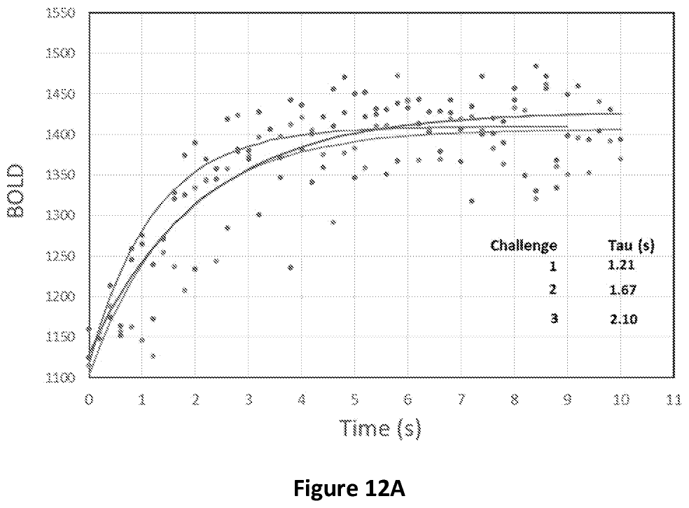

The processor 126 then fits the exponential function to the BOLD signal and calculates the time constant (τ) (see FIGS. 12 and 12A). Each time constant (τ) one LVEDV passes through the heart. The processor 126 is further configured to determine LVEDV. In some embodiments, LVEDV can be determined based on ultrasound data, MRI data, or CT data on the subject that is input via the user interface. In other embodiments, the memory 128 stores data representing average LVEDV values based on weight, height, sex, or age. The MRI 102 receives at the user interface 124, data representing at least one of the following characteristics of the subject 130: weight, height, sex, and age. The processor 126 estimates the LVEDV based on the average LVEDV values and the data representing the subject.

The processor 126 then calculates cardiac output (\dot{Q}) using the LVEDV and time constant (τ), discussed above, using Equation 12:

$$\dot{Q} = \frac{LVEDV}{τ} \qquad Equation 12$$

The processor 126 then calculates the shunt volume (SV) based on Equation 13, where heart rate (HR) is measured in beats per minute:

$$SV = \frac{\dot{Q}}{HR} \qquad Equation 13$$

Using the calculated SV, the processor 126 can calculate LVEF according to Equation 14:

$$LVEF = \frac{SV}{LDEDV} \qquad Equation 14$$

The processor 126 can calculate cardiac output (\dot{Q}) according to Equation 15:

\dot{Q}=SV×HR Equation 15

The system and method may be further used to characterize shunts in the atrium of the subject 130 caused by structural defects to the heart. In particular, patent foramen ovales, atrial septal defects (ASD), and ventricular septal defects (VSD) are known to cause left-to-right shunts in the heart. In one implementation, the MRI system 102 measures a BOLD signal in the pulmonary artery (SPA) or in the superior vena cava. The MRI system 102 may measure the BOLD signal over the duration of 1, 2, 3, or 4 heart beats by the subject 130. To improve the accuracy of the BOLD signal, the subject 130 may hold their breath for a period of time. In some examples, the subject 130 may hold their breath for a period of time lasting 1 to 10 heart beats.

The processor 126 may be configured to convert S_{PA} to [dOHb]_{PA}. The MRI 102 system measures the BOLD signal in the descending aorta (S_{ART}). (Note that in some implementations, the BOLD signal is measured in the left ventricle or the aortic arch instead of the descending aorta.) To improve the accuracy of the BOLD signal, the subject 130 may hold their breath for a period of time. In some examples, the subject 130 may hold their breath for a period of time lasting 1 to 10 heart beats.

Next, the SGD device 101 implements a change in alveolar PO₂ and the processor 126 calculates SaO₂ at the new PO₂. The processor can then convert SaO₂ to [dOHb]_{ART}. Next, the MRI system 102 can measure S_{ART}′. The processor 126 can calculate the fractional shunt by solving for x in the equations below.

[dOHb]_{PA}=(1−x)[dOHb]_{MV}+x([dOHb]_{ART}) Equation 16

[dOHb]_{PA′}=(1−x)[dOHb]_{MV}+x([dOHb]_{ART′}) Equation 17

[dOHb]_{PA}−[dOHb]_{PA′}=x([dOHb]_{ART}−[dOHb]_{ART}′) Equation 18

x=([dOHb]_{PA}−[dOHb]_{PA′})/[dOHb]_{ART}−[dOHb]_{ART′}) Equation 19

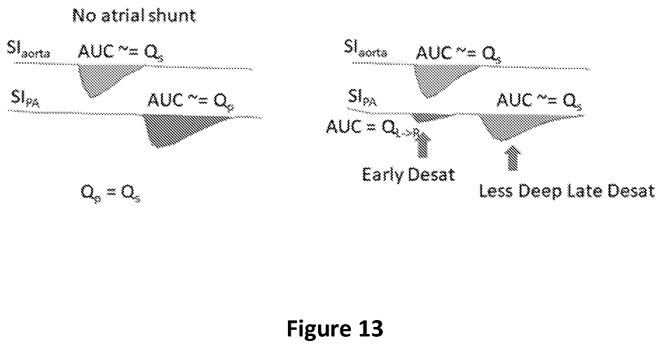

With reference to FIGS. 13, the system 100 may consider shunting as follows.

1. Signals SIₐₒᵣₜₐ, SI_{PA} may be collected from aorta and pulmonary artery (PA), respectively.

2. A square wave deoxygenation stimulus or arterial input function (AIF) may be administered. (Note: the same mechanism holds for a reoxygenation stimulus from a hypoxic baseline.)

3. The area under the curve (AUC) of the passage of the deoxygenated blood is determined by the mass of dOHb induced in the bolus.

4. Over time, the bolus will pass the pulmonary artery (PA). The AUC is same as in the aorta.

5. Assuming a left-to-right shunt (FIG. 13 at top right):

a. The artieral input function (AIF) is the same.

b. In left-to-right shunt (LR) some of pulmonary vein (PV) blood enters right ventricle (RV) and is seen in PA. The AUC of the shunted blood (AUC=Q_{L->R}) and the AUC of the delayed PA curve is the balance of the AIF.

Pulmonary blood flow (Qₚ) may be related to systemic blood flow (Qₛ) and the early oxygen desaturation (desat) area under the curve Q_{L->R} by Equation 20:

Qₚ=Qₛ+Q_{L→R} Equation 20

The ratio of pulmonary blood flow (Qₚ) to systemic blood flow (Qₛ) may be related to the AUCs in FIG. 13 by Equation 21:

$$\frac{{Q}_{p}}{{Q}_{s}} = \frac{( {Aorta}_{AUC} + Early {Desat}_{AUC} )}{{Aorta}_{AUC}} \qquad Equation 21$$

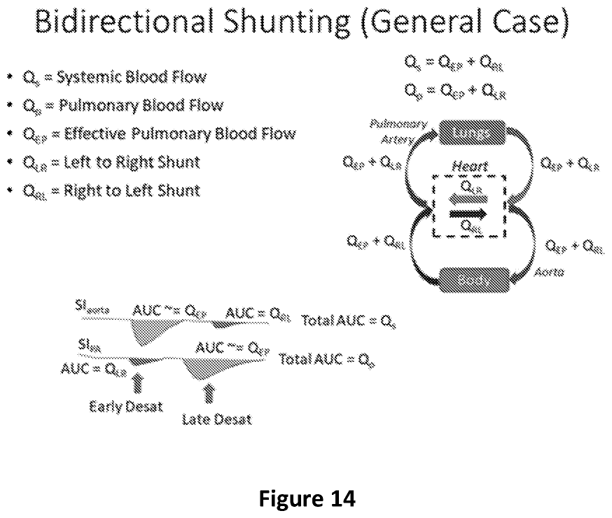

In other words, with reference to FIGS. 13 and 14, BOLD signals of the aorta and pulmonary artery (PA) may be monitored, starting from normoxia and during a hypoxic challenge. With no shunt, the hypoxia will transition into the aorta and, at a later time, it will be seen in the PA. However, if there is a cardiac shunt, the hypoxic challenge will be quickly seen in the PA. In the aorta, the main challenge would be seen a short time later. Hence, total AUC will be the sum of flow and shunt.

With reference to FIG. 14, the heart may be modelled as a box, in which shunting may occur in either direction. Atrial septal defects (ASDs) often have bidirectional shunting if they are longstanding or if they are large. Large ventricular septal defects (VSDs) often shunt left-to-right in systole and right-to-left in diastole.

The same principles apply with “early” desats and “late” desats in both the aorta and pulmonary artery. The AUCs are proportional to the flow. Bidirectional shunting introduces the consideration of “effective” pulmonary blood flow Q_{EP}, which is blood flow that has come from a systemic capillary bed directly to the pulmonary artery, as opposed to shunt flow. Isolated left-to-right and right-to-left shunts may be considered special cases of the generalized mathematical framework. That is, when Q_{R->L} is zero (i.e., a pure left-to-right shunt), then Qₛ=Q_{EP}. When Q_{L->R} is zero (i.e., a pure right-to-left shunt), then Qp=Q_{EP}.

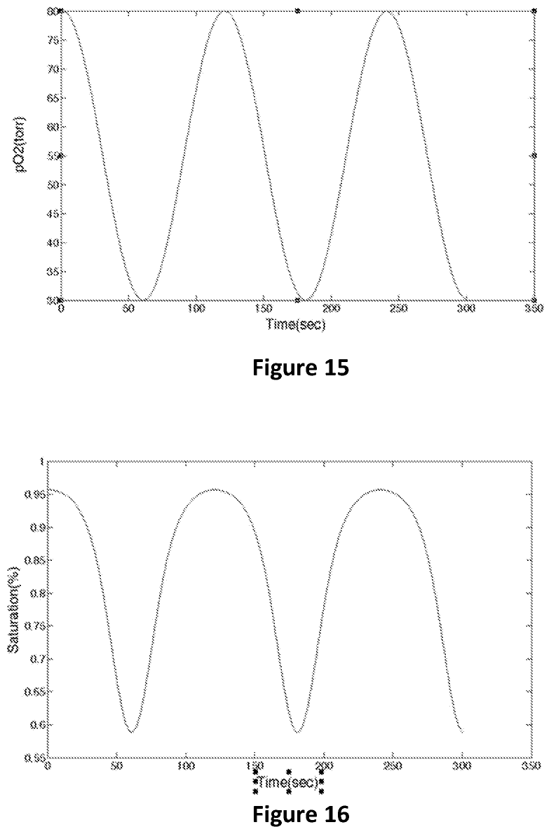

In various embodiments, the system 100 is configured to induce pulses of either desaturation or resaturation in the subject 130. However, in other embodiments, the system 100 can induce sinusoidal variations in oxygen saturation. FIGS. 15 to 21 show an embodiment where the system 100 induces sinusoidal variations in oxygen saturation. A sinusoidal variation in oxygen saturation may improve signal-to-noise ratio.

The processor 126 of the MRI system 102 may compute the CBF and MTT for each voxel based on the measured BOLD signal. Equation 22 shows the relationship between the measured BOLD signal (C), AIF, CBF, and the residual function R(t) at a given time (t).

C(t)=(CBF)AIF(t)R(t) Equation 22

In the above equation, the processor 126 can approximate R(t) according to Equation 23, where u(t) is the unit step function.

R(t)=e^{-t/MTT}u(t) Equation 23

The relationship is more simply represented in the Fourier domain, according to Equation 24:

C(ω)=(CBF)AIF(ω)R(ω) Equation 24

For a time-limited sinusoid, the processor 126 can compute AIF(ω) in Equation 22 as a pair of sine functions modulated by the sinusoidal frequency. The processor 126 can compute R(ω) according to Equation 25:

R(ω)=1/(1/MTT+jω) Equation 25

Alternatively, the processor 126 can estimate MTT from the phase delay between arterial voxels (in the common or internal carotid) and the tissue. The processor 126 can compute phase delay at the carrier frequency or as a weighted sum of frequencies (also called “group delay method”). Using the estimates of CBF and MTT calculates according to the above methods, the processor 126 can calculate CBV as the product of CBF and MTT.

Because the hemoglobin dissociation curve is nonlinear, implementing a sinusoidal cycle in PO₂ in the subject's lungs 130 does not implement a true sinusoid in arterial oxygen saturation. In one example, shown in FIG. 15, the processor 110 of the SGD device 101 may implement a sinusoidal variation in PO₂ from 40 to 90 torr. In this example, the subject 130 has a normal p50 of 26.4 torr. The predicted arterial saturation, shown in FIG. 16, is asymmetric with the positive oscillations being broader than the negative oscillations. FIG. 17 shows the observed BOLD signal intensity corresponding to the PO₂ in FIG. 15.

In order to implement sinusoidal variations in arterial oxygen saturation, two approaches may be taken.

Taking the first approach, the processor 110 limits the pO₂ oscillations to 30-50 torr. The hemoglobin dissociation curve is linear in this range and the resulting saturation waveform will be sinusoidal.

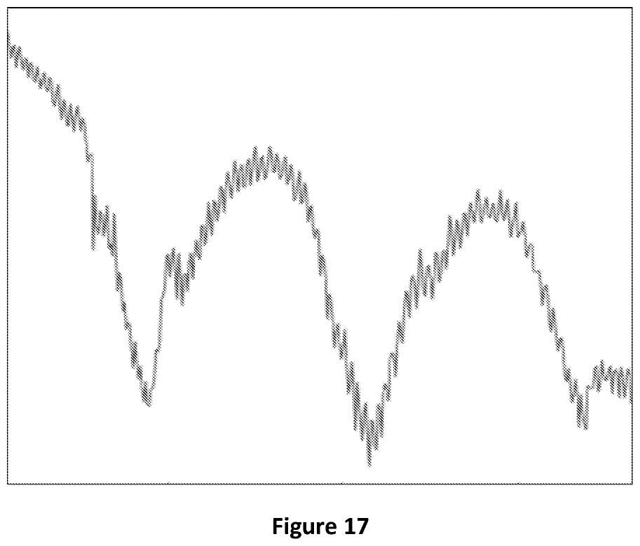

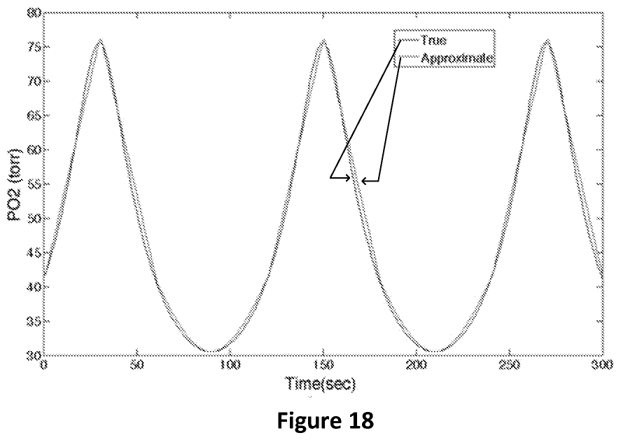

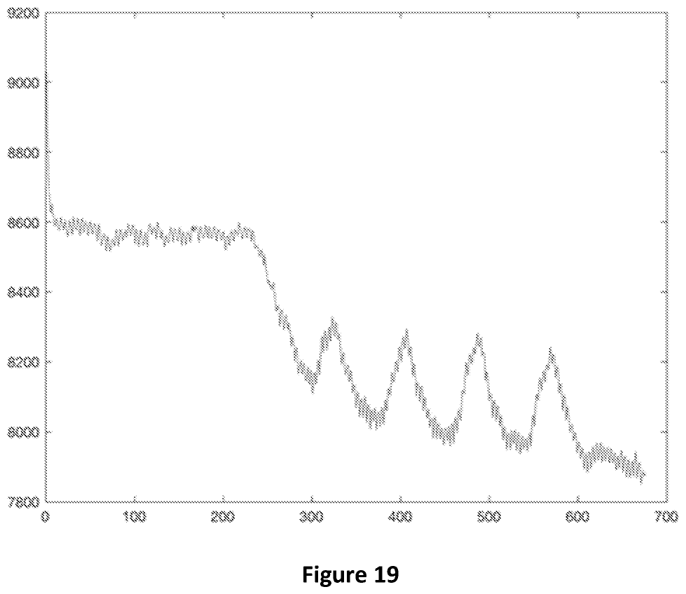

Taking the second approach, the processor 110 can use the Hill equation (or another suitable approximation to the hemoglobin dissociation curve shape) to calculate the pO₂ waveform necessary to achieve a sinusoidal fluctuation in oxygen saturation. FIG. 18 demonstrates the calculated pO₂ (“True”) necessary to achieve a sinusoidal saturation and an approximation to this curve using ramps and half-sinusoid stimuli. The “Approximate” curve is an approximation using ramps and half-sinusoids. FIG. 19 demonstrates the observed whole brain BOLD signal measured in the subject 130 in response to a 4-cycle sinusoid, similar to that shown in FIG. 18. Note that the positive and negative oscillations in FIG. 19 are not perfectly symmetric, however the area and period between the oscillations are much more balanced compared with the BOLD signal shown in FIG. 17. FIG. 19 also shows a superimposed exponential decline in signal intensity driven by the step change in average pO₂ between baseline and oscillating conditions. The processor 110 can reduce or eliminate this decline by exposing the subject 130 to several minutes of pO₂ at 40 torr to wash out excess oxygen from the lung of the subject 130.

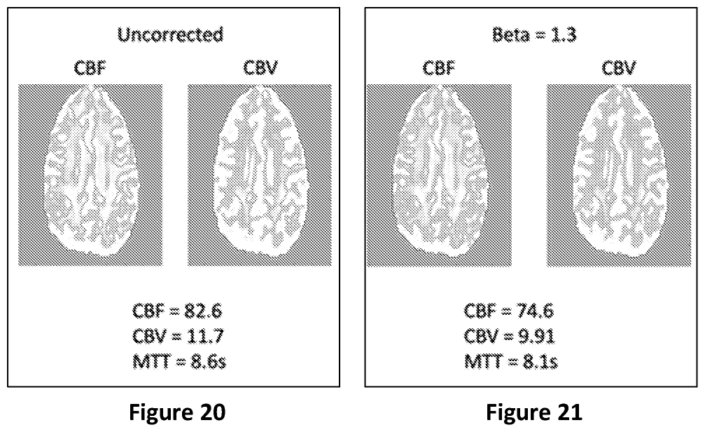

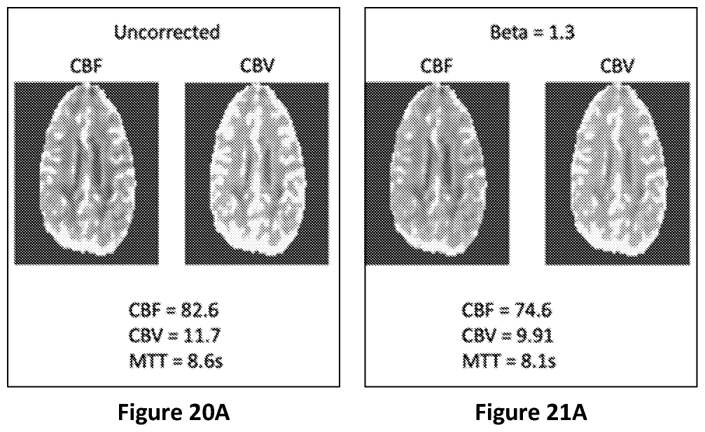

FIGS. 20 and 21 show brain maps of the CBF and CBV calculated by the processor 126 of the MRI system 102 using the pO₂ waveform from FIG. 18. In FIG. 20, the brain maps have not been corrected for to account for the nonlinear dissociation curve of hemoglobin. Both tissue and blood regions of interest were assumed to vary linearly with blood oxygen saturation. Fluctuations in the sagittal sinus signal are used as a surrogate for the AIF, however, since the sagittal sinus is a large vein with 100 percent volume fraction of blood, it may experience greater signal loss with desaturation than tissue exposed to the same oxygen saturation. In FIG. 21, the brain maps have been corrected based on the assumption that blood has a quadratic relationship with oxygen saturation and tissue varied with an exponent of 1.3.

One advantage of using sinusoidal variation in oxygen saturation is an improved signal-to-noise ratio. Another advantage is that physiological noise is suppressed by the use of a single-input-single-output frequency. A third advantage is that it is easy to estimate MTT using the phase of the Fourier transformation at the carrier frequency. A further advantage is that sinusoidal stimuli are fairly innocuous and tolerable by the subject 130.

In view of the above it is contemplated that, when considering the relationship between hypoxia and blood flow, it is known that resting blood flow can increase with decreasing oxygenation leading to a falsely elevated resting blood flow. A 50% drop in the partial pressure of arterial oxygen saturation can result in a 15% increase in resting CBF. However, following an abrupt reduction on O₂ saturation, there is a reported delay of approximately 3 minutes before this blood flow increase occurs. The rapid 10-15 second return to normoxia during the oxygen bolus protocol that occurs during this 3-minute delay is expected help to mitigate this flow response, thus leaving resting flow unaltered.

The paramagnetic effects of intravascular gadolinium and dHb are expected to have a similar non-linear behavior and differences are expected. Geometric effects of the signal properties may be altered by differences in compartmentalization where gadolinium is extracellular and dHb is intracellular, although this is expected to have a minimal effect.

For the duration after the formation of dOHb from OHb, or formation of OHb from dOHb in the Pulmonary vein, the [dOHb] can change as a result of admixture of blood in the pulmonary vein from the pulmonary artery (PA) via arterio-venous anastomoses, interatrial connections, patent ductus arteriosus, intraventricular shunt, and attenuation due to metabolism of the tissues at the level of the microcirculation giving up oxygen. These are confounders. However, if anticipated, the techniques discussed herein can be used to diagnose and quantify such changes. Examples of identifying intracardiac shunts have been discussed above. The metabolism of the tissues during the transit of blood does not affect precapillary [dOHb] and thus it can be used as an arterial contrast agent. This does not happen with gadolinium. Another advantage of [dOHb] over gadolinium-based compounds as a contrast agent is that [dOHb] remains intravascular whereas gadolinium may diffuse intracellularly and past the blood-brain barrier. However, [dOHb] is expected to behave substantially identically to gadolinium as long as brain oxygen consumption does not change during the bolus of [dOHb].

In addition, measurement of flow metrics using an arterial input function is often required to generate the three major flow metrics using deconvolution methods. At issue is the volume averaging of structures adjacent to small intra-cerebral arteries reducing the magnitude of the AIF resulting in the calculation of higher than normal blood flow. This can be mitigated using smaller voxel sizes. The data showed that little is gained after the first challenge, reducing image acquisition to about only 2 minutes.

Further, the signal-to-noise ratio may be improved by inducing a sinusoidal paradigm.

It will now be apparent to the skilled person that there are a number of advantages provided by the above disclosed method. The ability to rapidly and precisely control arterial oxygenation of hemoglobin in the form of oxygenated hemoglobin (OHb) boluses while maintaining isocapnia provides the means to map cerebral blood flow metrics during BOLD MRI without changing cerebral blood flow. The values reported are comparable to those obtained with gold standard PET imaging flow measurements in healthy individuals. Furthermore, the method disclosed overcomes some of the limitations of existing perfusion imaging methodologies. Most methods utilize intra-venous bolus administration of tracers (contrast agents) that are then observed as they pass from supply arteries to the tissues and then on into draining veins and dural sinuses enabling calculation of primary perfusion metrics including CBV, CBF, and MTT. These metrics have been used to obtain important information that can aid in the characterization of cerebrovascular and other brain disorders.

As compared with intra-venously administered contrast agents, the above disclosed methods are non-invasive (i.e. needle free). Additionally, the method provides more accurate measurements since deoxyhemoglobin is an endogenous contrast agent that is generated in the lungs, reducing delay and dispersion of the tracer bolus. Moreover, deoxyhemoglobin eliminates the use of expensive tracers and associated adverse effects including contrast reactions and potential organ injury in the case of iodinated contrast (renal dysfunction). Finally, deoxyhemoglobin permits an unlimited number of follow-up studies as there is no ionizing radiation from radioactive tracers or imaging devices using x-rays.

Furthermore, precise repeatability enables the generation of normal mean and range of tests in a population. This enables the scoring of tests in a single patient or subject with respect to normality. Precisely repeatable stimuli and baseline levels may be used to generate an atlas of flow values for healthy people or any subpopulation and the assessment of blood flow in any single subject compared to one of the cohorts.

The many features and advantages of the invention are apparent from the detailed specification and, thus, it is intended by the appended claims to cover all such features and advantages of the invention that fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

1. A method comprising:
generating a change in deoxyhemoglobin in a subject;
conducting magnetic resonance imaging on the subject; and
using the deoxyhemoglobin of the subject as a contrast agent for a weighted imaging of the magnetic resonance imaging.

2. The method of claim 1, further comprising synchronizing the level of deoxyhemoglobin with data of the magnetic resonance imaging.

3. The method of claim 1, further comprising controlling one or both of breathing rate and gas composition to exhibit different temporal and/or localized responses in the level of deoxyhemoglobin in the subject during the magnetic resonance imaging.

4. The method of claim 1, wherein the weighted imaging comprises a weighting imaging (T2*) of a transverse relaxation time (T2).

5. The method of claim 1, wherein generating the change in the deoxyhemoglobin in the subject comprises varying a partial pressure of oxygen in the lungs of the subject.

6. The method of claim 1, further comprising using a single or multiple gradient-echo for contrast preparation and a single-shot signal indicative of a dynamic change of deoxyhemoglobin in response to a rapid and controlled change in oxygen concentration provided for inhalation by the subject.

7. The method of claim 1, further comprising using single or multiple spin-echo contrast preparation and a single-shot signal to detect a weighted change in a magnetic resonance imaging signal caused by a change in deoxyhemoglobin to measure blood flow, blood volume, transit time, or a combination of such.

8. The method of claim 1, further comprising using a combination gradient echo and spin echo for contrast preparation and a single shot signal indicative of mixed T2 and T2*-weighted changes in a magnetic resonance imaging signal caused by a change in deoxyhemoglobin to measure blood flow, blood volume, transit time, or a combination of such.

9. The method of claim 1, further comprising deriving from a magnetic resonance imaging signal responsive to a change in deoxyhemoglobin a peak signal change, an onset, a time to peak, a full width half maximum, a recovery half time, an area under the curve, or a combination of such.

10. The method of claim 1, further comprising applying a Fourier analysis to a characteristic of a magnetic resonance imaging signal to define a set of voxels.

11. The method of claim 10, further comprising applying the Fourier analysis to generate a generate map of an arterial transit time, a capillary transit time, a venous transit time, or a combination of such for use in diagnosis of an arteriovenous fistula, a collateral vessel, or both.

12. The method of claim 10, further comprising applying time-delay information from a phase map of the Fourier analysis to form a static visualization of vasculature.

13. The method of claim 10, further comprising applying time-delay information from a phase map of the Fourier analysis to form a static visualization of vasculature.

14. The method of claim 10, further comprising applying time-delay information from a phase map of the Fourier analysis to output a video of a dynamic contrast change as contrast passes continuously among different vascular levels.

15. The method of claim 1, further comprising computing a perfusion quantity based on a response to a bolus inspiration that changes the deoxyhemoglobin in the subject.

16. The method of claim 15, wherein the perfusion quantity comprises a cerebral blood flow (CBF), a cerebral blood volume (CBV), a mean transit time (MTT), an arterial arrival time (ATT), or a combination of such.

17. The method of claim 1, further comprising computing an Arterial Input Function (AIF).

18. The method of claim 1, further comprising determining a capillary transit time heterogeneity (CTH) with reference to a distribution of transit time within a region or voxel of a signal of the magnetic resonance imaging.

19. The method of claim 1, further comprising computing a performance status of the left ventricle of the subject, wherein the performance status comprises a cardiac output (\dot{Q}), a stroke volume (SV), or a left ventricular ejection fraction (LVEF).

20. Use of deoxyhemoglobin of the subject as a contrast agent in magnetic resonance imaging.

21. A method of controlling deoxyhemoglobin in a subject, the method comprising:
providing a gas for the subject to inhale to obtain a target lung partial pressure of oxygen and a target lung partial pressure of carbon dioxide to obtain a target level of deoxyhemoglobin in the subject's blood.

22. The method of claim 21, further comprising using a sequential gas delivery apparatus to deliver the gas to the subject.

23. The method of claim 21 or 22, wherein the target level of deoxyhemoglobin is arterial.

24. The method of claim 21 or 22, wherein the target level of deoxyhemoglobin is venous.

25. The method of any of claims 21 to 24, wherein providing the gas for the subject to inhale causes a rapid change in lung partial pressure of oxygen and lung partial pressure of carbon dioxide to cause a rapid change in deoxyhemoglobin.

26. The method of any of claims 21 to 25, further comprising using dynamic end-tidal forcing to obtain one or both of the target lung partial pressure of oxygen and the target lung partial pressure of carbon dioxide.

27. The method of any of claims 21 to 26, further comprising prospective targeting of the target lung partial pressure of oxygen independent of breath volume and frequency.

28. The method of any of claims 21 to 27, further comprising prospective targeting of the target lung partial pressure of carbon dioxide independent of breath volume and frequency.

29. The method of claim 21, further comprising controlling one or both of breathing rate and gas composition to obtain durations of stimulus and baseline levels of deoxyhemoglobin.

30. The method of claim 29, further comprising obtaining durations of stimulus and baseline levels of deoxyhemoglobin for a plurality of subjects to generate an atlas.

31. Use of hypoventilation and/or breath holding for a subject to generate deoxyhemoglobin in the subject for use as contrast agent in magnetic resonance imaging.

32. A method of calibrating magnetic resonance imaging, the method comprising:
controlling blood deoxyhemoglobin in a subject by administering a gas that provides a lung partial pressure of oxygen and a lung partial pressure of carbon dioxide to the subject;
capturing a calibrating magnetic resonance imaging signal while controlling the blood deoxyhemoglobin in the subject;
obtaining a relationship of the blood deoxyhemoglobin to the calibrating magnetic resonance imaging signal; and
applying the relationship to a subsequent magnetic resonance imaging signal for a tissue to obtain tissue oxygenation information.

33. The method of claim 32, comprising administering the gas to provides different levels of lung partial pressure of oxygen and lung partial pressure of carbon dioxide.

34. The method of claim 32, wherein the calibrating magnetic resonance imaging signal is obtained from the subject's aorta.

35. The method of claim 32, wherein the calibrating magnetic resonance imaging signal is obtained from the subject's vena cava or right atrium.
